(19) United States
(12) Patent Application Publication
(10) Pub. No.: US 2002/0111318 A1
(43) Pub. Date: Aug. 15, 2002

(54) Process for the preparation of non-hygroscopic azithromycin dihydrate

(76) Inventor: Srinivasan Rengaraju, Vadodara (IN)

Correspondence Address: STAAS & HALSEY LLP, 700 11TH STREET, NW, SUITE 500, WASHINGTON, DC, 20001, US

(21) Appl. No.: 10/050,897

(22) Filed: Jan. 18, 2002

(30) Foreign Application Priority Data
Jan. 29, 2001 (IN) 95/MUM/2001

(51) Int. Cl. A61K 31/7048; C07H 17/08

(52) U.S. Cl. 514/29; 536/7.4; 536/17.4

(57) ABSTRACT

An improved process for preparing non-hygroscopic Azithromycin dihydrate wherein Azithromycin monhydrate can be converted to Azithromycin dihydrate with continous stirring/agitation in presence of a mixture of at least one solvent and water until non-hygroscopic crystals of Azithromycin dihydrate are obtained. The solvent used in the process can be selected from the group comprising dimethylformamide, dimethylacetamide, acetonitrile and iso-propanol.

### CROSS-REFERENCE TO RELATED APPLICATIONS

This application is based upon and claims priority of Indian Patent Application No. 95/Mum/2001 filed Jan. 29, 2001, the entire contents of same being incorporated herein by reference.

### BACKGROUND OF THE INVENTION

1. Field of the Invention

The invention relates to an improved process for the production of non-hygroscopic Azithromycin dihydrate.

2. Description of the Related Art

Azithromycin (1) (USAN generic name for 9-Deoxo-9a-aza-9a-methyl-9a-homo-Eryhromycin A) is a 15 membered ring macrolide belonging to a new class of antibiotics termed as “Azalides”, due to the incorporation of nitrogen atom in the macrocyclic ring. It is derived from the 14-membered macrolide antibiotic Erythromycin A and shows significant improvement in its activity against gram negative organisms compared to Erythromycin A (C J Dunn and L B Barradell Azithromycin: A Review of its Pharmacological properties and use as a 3-day therapy in respiratory tract infections, Drugs, 1996 (March,51(3)483-505).

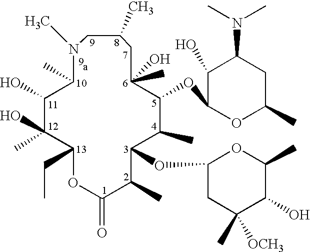

Azithromycin was first discovered by G. Kobrehel and S. Djokic (Belgium Patent No. 892357 and its related U.S. Pat. No. 4,517,359). S. Djokic et al (J CHEMRESEARCH(S). 1988, 132 and idem miniprint 1988, 1239), have demonstrated the existence of the dihydrate form of Azithromycin.

In U.S. Pat. 4,517,359, Azithromycin was isolated by evaporation of its chloroform solution under vacuum. A melting point of 113-115° C. was reported for this preparation. There is no mention of the crystal form of Azithromycin in this patent. In all probability, it is an amorphous powder.

Azithromycin preparation was also described by G. M. Bright in U.S. Pat. No. 4,474,768 (with corresponding E.P. No. 0101186), wherein the amorphous azithromycin foam obtained by evaporation of a methylene chloride solution was crystallized from ethanol: water to give Azithromycin melting at 142° C. In this patent, there also is no mention of the crystal form of Azithromycin.

Azithromycin is known to exist in two crystalline forms. In the European Patent 0298650 (with corresponding Indian Patent IN 168896), Allen and Nepveux have shown that Azithromycin crystallized from ethanol : water gives a hygroscopic monohydrate form which melts at 142° C. They have described a method for conversion of the hygroscopic monohydrate form to the dihydrate form of Azithromycin. This involves recrystallization from a mixture of solvents containing tetrahydrofuran and an aliphatic (C₅-C₇) hydrocarbon in presence of water. The Azithromycin dihydrate thus prepared has a melting point of 126° C. The process of Allen et al suffered from some disadvantages. In this process a mixture of tetrahydrofuran and a hydrocarbon like hexane is used. Mixtures of two organic solvents result in higher recovery costs and besides handling of hydrocarbon solvents requires extra care due to fire hazards.

S. Djokic et al (Journal of Chemical Research (S) 1998, 132 and idem miniprint, 1998, 1239) also have demonstrated the existence of the dihydrate form of Azithromycin.

Bayod Jasanda et al in U.S. Pat. 5,869,629 have disclosed a method of preparation of Azithromycin dihydrate by the recrystallization of the hygroscopic form of Azithromycin from acetone : water and agitating the slurry for 24 hrs. Again this method has inherent disadvantages. The conversion of monohydrate to dihydrate form requires stirring for long periods such as for 24 hours.

European Patent EP 0941999 also describes the crystallization of azithromycin first by dissolving azithromycin in aqueous acetone and then precipitating as dihydrate by adjusting pH to alkaline side.

The monohydrate form of Azithromycin is difficult to handle during its formulation into capsules or other forms due to its hygroscopicity. Hence the stable dihydrate form is used in the formulations of Azithromycin. Due to this importance of Azithromycin dihydrate in formulations of azithromycin, methods of conversion of unstable monohydrate form to stable dihydrate form is required.

### SUMMARY OF THE INVENTION

It is therefore an objective of the invention to provide an improved process for the production of non-hygroscopic Azithromycin dihydrate.

It is a further object of the present invention to provide a Azithromycin dihydrate employing selected solvents whereby the process can be carried out at room temperature with no additional energy input.

It is a further objective of the invention to provide a process for the production of Azithromycin dihydrate wherein the process is quick and formation of crystals can be easily observed.

These and other objects of the invention that will now be described in the embodiments of the specification.

The invention discloses an improved process for preparing non-hygroscopic Azithromycin dihydrate which comprises:

(a) preparing a suspension of Azithromycin monohydrate in any conventional manner; and

(b) subjecting the suspension of Azithromycin monohydrate to stirring/agitation in presence of a mixture of at least one solvent such as herein described and water till non-hygroscopic crystals of Azithromycin dihydrate are obtained.

The solvent in the process of the invention can be selected from the group comprising dimethylformamide, dimethylacetamide, acetonitrile and iso-propanol. The crystals of Azithromycin dihydrate are subjected to filtration and drying in any conventional manner.

### BRIEF DESCRIPTION OF THE DRAWINGS

FIG. 1 is a graph illustrating the characteristic solid state IR spectrum (KBr pellet) of azithromycin monohydrate;

FIG. 2 is a graph illustrating the characteristic solid state IR spectrum (KBr pellet) of azithromycin dihydrate;

FIG. 3 is a graph illustrating the characteristic X-Ray diffraction pattern of azithromycin dihydrate; and

FIG. 4 is a graph illustrating the characteristic X-Ray diffraction pattern of azithromycin monohydrate.

### DETAILED DESCRIPTION OF THE PREFERRED EMBODIMENTS

The monohydrate form of Azithromycin is difficult to handle during its formulation into capsules or other forms due to its hygroscopicity. Hence the stable dihydrate form is used in the formulations of Azithromycin. Due to this importance of Azithromycin dihydrate in formulations of azithromycin, methods of conversion of unstable monohydrate form to stable dihydrate form is desired.

The present invention discloses a simple and novel method for the conversion of hygroscopic monohydrate form of azithromycin to the dihydrate form of azithromycin. The inventors have found due to the experimentation done by them that the hydrosocpic monohydrate of azithromycin can be converted to the stable dihydrate form by agitating a slurry of monohydrate form in a water-solvent mixture.

The solvent which is employed in the process of the invention is selected from the group consisting of (1) dimethyl formamide (2) dimethyl acetamide (3) acetonitrile (4) iso-propanol. The agitation of the slurry is carried out at ambient temperature without the requirement of heating or cooling the mixture. The transformation of the azithromycin monohydrate crystal to azithromycin dihydrate crystal can be easily followed by observing the crystal slurry under the microscope, as the crystal habit of both the forms are different.

The monohydrate crystals are of cubic habit which during the agitation in aqueous solvent mixture slowly gets converted to the dihydrate form. The crystals of dihydrate are of a rhombic habit which are easily distinguishable under the microscope from the cubic habit of the monohydrate, thus making the process amenable for quick and easy process control. When all the crystals are of rhomboid type, the agitation is stopped and the slurry is filtered and dried under vacuum. The stirring is usually carried out for 2-18 hrs. by which time the transformation of the monohydrate to dihydrate takes place.

Preferably the ideal water-to-solvent ratio is 1:1 and the slurry concentration is kept at 50%, so as to ensure maximum recovery of the dihydrate. Lower concentrations of water in the solvent is not contraindicated for the conversion but it is avoided as loss by solubility of Azithromycin in such systems will increase.

The agitation of the slurry of Azithromycin monohydrate in solvent: water mixture can be carried out by conventional methods of agitation, such as magnetic stirring in the laboratory scale or mechanical agitation as practiced in industrial scale.

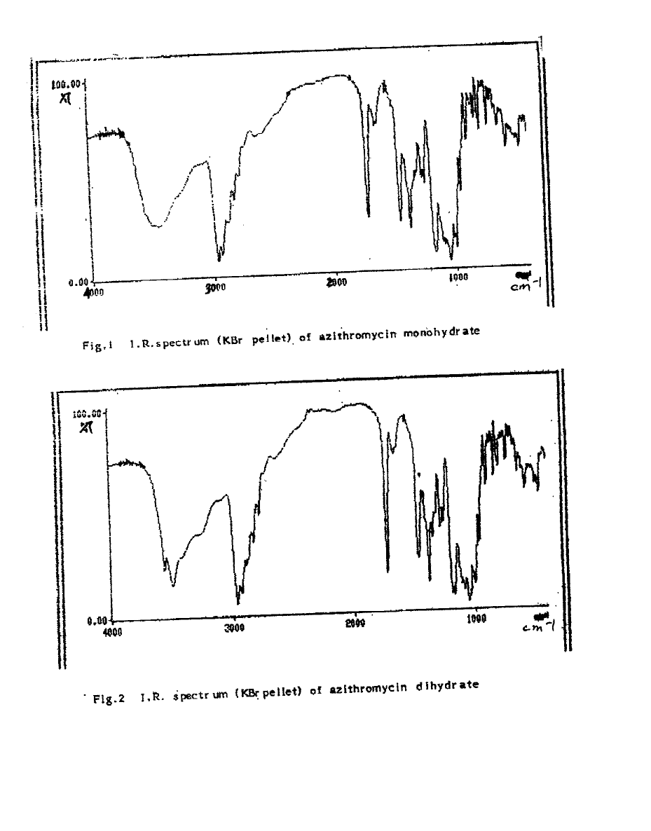

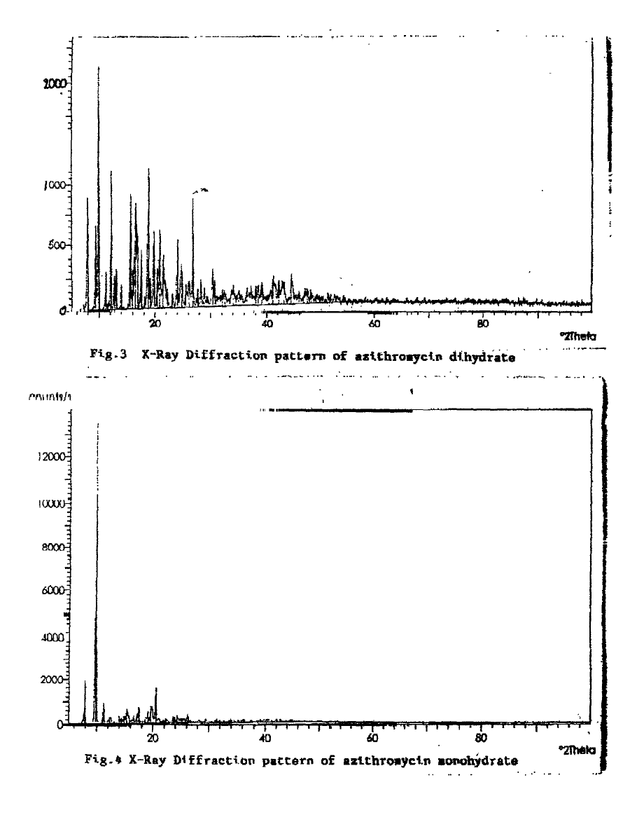

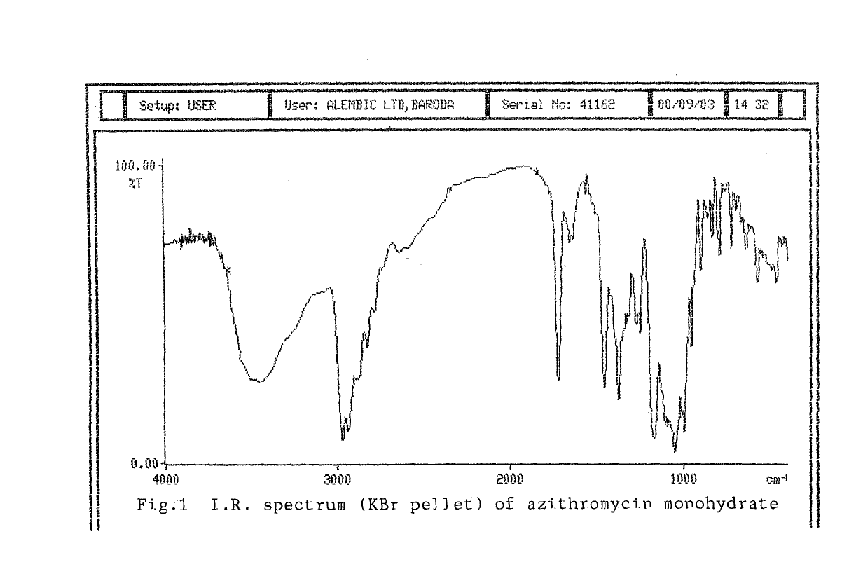

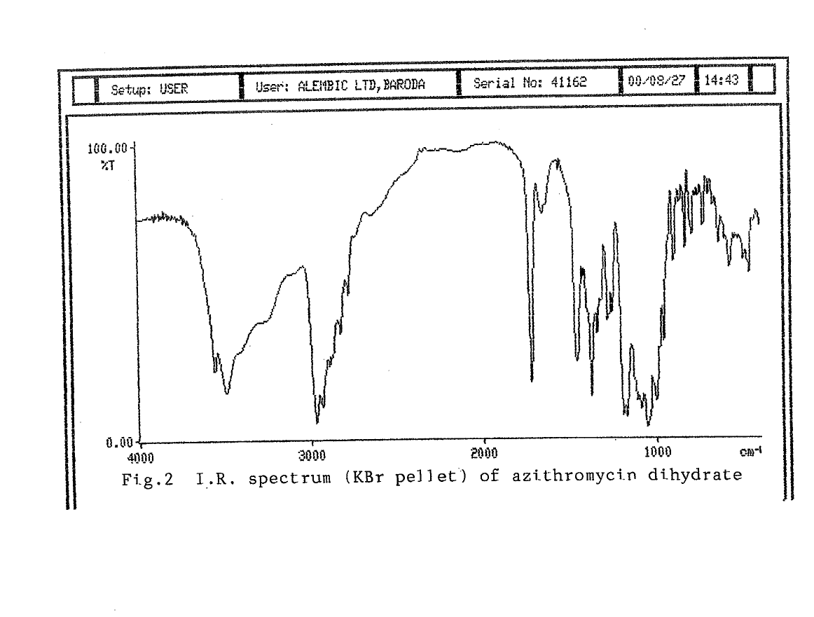

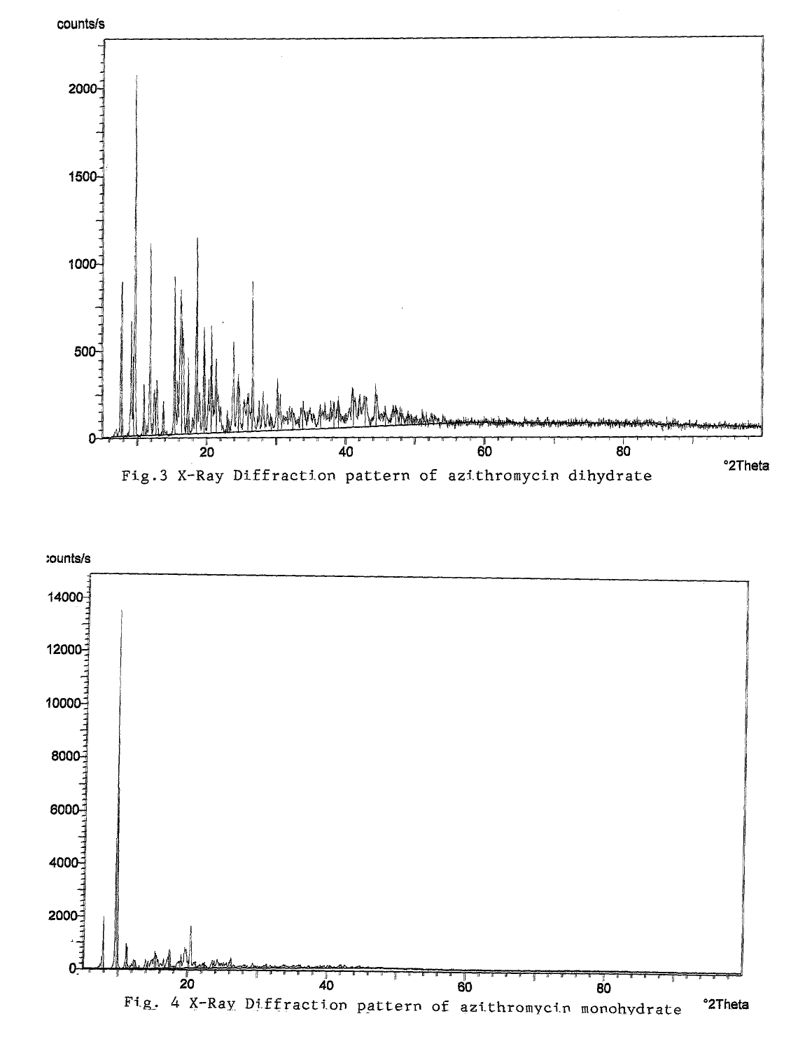

The Azithromycin dihydrate is easily distinguished from the monohydrate by their characteristic solid state (KBr, pellet) IR spectra, such as illustrated in FIGS. 1 and 2. The monohydrate shows a broad peak in the hydroxyl stretching region at 3450 cm⁻¹ (broad) (FIG. 1), whereas the dihydrate shows two peaks in this region at 3560 cm⁻¹ (shoulder) and 3495 cm⁻¹ (FIG. 2). There are also characteristic absorption for the two forms in the C-0, C-N stretching regions (1000-1200 cm⁻¹) (FIGS. 1 & 2). The two forms are also distinguished by their characteristic x-ray diffraction patterns, such as illustrated in FIGS. 3 and 4.

Unlike any of the prior patents or methods, the invention provides the choice of using any of the four solvents—dimethyl formamide, dimethyl acetamide, acetonitrile or iso-propanol—in the process. As the process is carried out at ambient temperature, no additional energy input is needed. Moreover, the process can be easily and quickly followed by observing the crystal habit under a microscope. Therefore, it is possible to terminate the agitation (stirring) at optimum time. Azithromycin is produced by the reductive methylation of 9-Deoxo-9-α-aza-9a-homoerythromycin using formaldehyde-formic acid mixture. The reaction generates certain impurities which can be removed in the aqueous-solvent slurrying step of the monohydrate to dihydrate conversion.

The invention will now be described with reference to the following examples which are only illustrative and should in no way be understood to limit the scope of the invention in any manner whatsoever.

Preparation of Hygroscopic Azithromycin Monohydrate

9-Deoxo-9a-aza-9a-homoerythromycin A (73.5 g-0.1 mole) was dissolved in 250 ml acetone. To this solution, formic acid (19 ml) followed by formaldehyde (37%, 20 ml) were added and refluxed for 24 hrs. The pH of the reaction mixture was adjusted with alkali to 10.5 and filtered to remove particles. To the filtered acetone solution equal volume of water was added to precipitate azithromycin hygroscopic monohydrate as cube shaped crystals. The crystals were filtered and dried under vacuum at 50° C. to give 65 g of azithromycin monohydrate melting at 130-131° C. having a water content of 3.42% (by Karl Fischer titration method). This sample of hygroscopic monohydrate has a characteristic solid state (KBr pellet) IR spectrum (FIG. 1) and a characteristic x-ray diffraction pattern (FIG. 4). The crystals absorbed moisture on exposure to ambient atmosphere and a moisture content of 5.4% was reached in 48 hrs.

Example 1

### Preparation of Azithromycin Dihydrate from Hygroscopic Azithromycin Monohydrate Using Iso-propanol: Water Mixture.

10 gms of hygroscopic azithromycin-monohydrate was suspended in a mixture of iso-propanol (10 ml) and water (10 ml) and stirred at ambient temperature. The transformation of cubical crystals of monohydrate form to the rhomboid form crystals of dihydrate was followed by checking the crystal habit under a microscope at every two hour interval. At 16 hours the rhomboid dihydrate crystals only were seen. The slurry was filtered and dried under vacuum at 50° C. to give 9.8 g of azithromycin dihydrate. It had a melting point of 126-128° C. and water content of 4.65% (Theoretical 4.586) (by Karl-Fischer titration method). It has a characteristic solid state IR spectrum (KBr pellet) (FIG. 2) and x-ray diffraction pattern (FIG. 3). On exposure to ambient atmosphere there was no change in the moisture content of the dihydrate crystals.

Example 2

### Preparation of Azithromycin Dihydrate from Hygroscopic Azithromycin Monohydrate Using Acetonitrile : Water Mixture.

10 gms of hygroscopic azithromycin-monohydrate was suspended in a mixture of acetonitrile (10 ml) and water (10 ml) and stirred at ambient temperature. The transformation of cubical crystals of monohydrate form to the rhomboid form crystals of dihydrate was followed by checking the crystal habit under a microscope at every two hour interval. At 8 hours the rhomboid dihydrate crystals only were seen. The slurry was filtered and dried under vacuum at 50° C. to give 9.8 g of azithromycin dihydrate. It had a melting point of 126-128° C. and water content of 4.68% (Theoretical 4.586) (by Karl-Fischer titration method). It has a characteristic solid state IR spectrum (KBr pellet) (FIG. 2) and x-ray diffraction pattern (FIG. 3). On exposure to ambient atmosphere there was no change in the moisture content of the dihydrate crystals.

Example 3

### Preparation of Azithromycin Dihydrate from Hygroscopic Azithromycin Monohydrate Using Dimethyl Formamide: Water Mixture.

10 gms of hygroscopic azithromycin-monohydrate was suspended in a mixture of dimethyl formamide (10 ml) and water (10 ml) and stirred at ambient temperature. The transformation of cubical crystals of monohydrate form to the rhomboid form crystals of dihydrate was followed by checking the crystal habit under a microscope at hourly interval. At 3 hours the rhomboid dihydrate crystals only were seen. The slurry was filtered and dried under vacuum at 50° C. to give 9.8 g of azithromycin dihydrate. It had a melting point of 126-128° C. and water content of 4.6% (Theoretical 4.586) (by Karl-Fischer titration method). It has a characteristic solid state IR spectrum (KBr pellet) (FIG. 2) and x-ray diffraction pattern (FIG. 3). On exposure to ambient atmosphere there was no change in the moisture content of the dihydrate crystals.

Example 4

### Preparation of Azithromycin Dihydrate from Hygroscopic Azithromycin Monohydrate Using Dimethyl Acetamide: Water Mixture.

10 gms of hygroscopic azithromycin-monohydrate was suspended in a mixture of dimethyl acetamide (10 ml) and water (10 ml) and stirred at ambient temperature. The transformation of cubical crystals of monohydrate form to the rhomboid form crystals of dihydrate was followed by checking the crystal habit under a microscope at every two hour interval. At 4 hours the rhomboid dihydrate crystals only were seen. The slurry was filtered and dried under vacuum at 50° C. to give 9.8 g of azithromycin dihydrate. It had a melting point of 126-128° C. and water content of 4.63% (Theoretical 4. 586) (by Karl-Fischer titration method). It has a characteristic solid state IR spectrum (KBr pellet) (FIG. 2) and x-ray diffraction pattern (FIG. 3). On exposure to ambient atmosphere there was no change in the moisture content of the dihydrate crystals.

Although preferred embodiments of the present invention have been shown and described, it will be appreciated by those skilled in the art that changes may be made in these embodiments without departing from the principle and spirit of the invention, the scope of which is defined in the appended claims and their equivalents.

What is claimed is:

1. A process for preparing non-hygroscopic Azithromycin dihydrate comprising:
preparing a suspension of Azithromycin monohydrate; and
subjecting the suspension of Azithromycin monohydrate to stirring/agitation in presence of a mixture of at least one solvent and water until non-hygroscopic crystals of Azithromycin dihydrate are obtained.

2. A process as claimed in claim 1, wherein the said solvent is selected from the group comprising dimethylformamide, dimethylacetamide, acetonitrile and iso-propanol.

3. A process as claimed in claim 1, wherein the crystals of Azithromycin dihydrate are subjected to filtration and drying.
